(19) United States
(12) Patent Application Publication
(10) Pub. No.: US 2007/0140973 A1
(43) Pub. Date: Jun. 21, 2007

(54) CONTRAST AGENTS FOR MYOCARDIUM PERFUSION IMAGING

(75) Inventors: D. Scott Edwards, Bedford, MA (US); David S. Casebier, Carlisle, MA (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(73) Assignee: Bristol-Myers Squibb Pharma Company

(21) Appl. No.: 11/610,216

(22) Filed: Dec. 13, 2006

Related U.S. Application Data
(60) Provisional application No. 60/750,654, filed on Dec. 15, 2005.

(51) Int. Cl. A61K 49/10

(52) U.S. Cl. 424/9.321

(57) ABSTRACT

The present invention is directed, in part, to compounds and methods for diagnostic imaging, comprising administering to a patient a contrast agent which has an overall negative charge.

### CROSS-REFERENCE TO RELATED APPLICATION

This present application is related to U.S. Provisional Application Ser. No. 60/750,654 filed Dec. 15, 2005, the contents of which are incorporated by reference herein in their entirety.

### FIELD OF THE INVENTION

The present invention relates to novel compounds comprising imaging moieties, and their use for diagnosing certain disorders in a patient.

### BACKGROUND OF THE INVENTION

Contrast agents are used in diagnostic imaging of an individual. A typical contrast agent has both targeting and reporting capabilities, the former for causing accumulation of the contrast agent in predetermined tissues of interest and the latter to allow imaging of the area of contrast agent accumulation. It is especially desirable to develop contrast agents which maximize retention in the tissues of interest.

One area of particular interest is imaging myocardial reperfusion and ischemia. During myocardial reperfusion and ischemia, the immune system is activated through various mechanisms, including the complement pathways Recent studies have also shown that the structure of the plasma membrane of cardiomyocytes in ischemic tissue “flip-flops”, presenting a relatively high amount of phosphatidylcholine on the surface of the cell.

It is postulated that this promotes the binding of C-reactive protein (CRP) to the compromised tissue. The CRP in turn can activate complement and form complexes that are found in elevated levels of fatally infarcted human myocardium (Lagrand et al. Circulation 1997 (95) 97-103). It has been shown that human CRP increases infarction size in a complement-dependent fashion in a rat model of acute myocardial infarction (Griselli et al, J Exp Med. 1999 (190), 1733-1739). The CRP-complement complex in turn has been implicated in the recruitment of neutrophils. The action of the neutirophils leads to cell damage (Jordan, et al. Cardiovascular research 1999, (43), 860-878).

The incorporation of anionic lipids into the surface of a cell or particle increases the uptake of components involved in the complement membrane attack complex (C5b-C7, Liu, et al. Blood 1999, (93), 2297-2301). This uptake can promote the chemotactic affinity to C5a, predominant in the monocyte enrichment of ischemic myocardium- 1-4 hours post reperfusion (Birdsall, et al. Circulation. 1997, 95(3), 684-92).

Recent studies have shown that molecules having an overall negative charge are better retained in the microvasculature of the myocardium, having retention times that are significantly longer than uncharged molecules.

It is an object of the present invention to exploit these developments for use in myocardial diagnostic imaging techniques. The present invention is directed to these, as well as other important ends.

### SUMMARY OF THE INVENTION

In one embodiment, the present invention includes methods and compositions for imaging myocardium perfusion, comprising providing contrast agents described herein having an overall negative charge, administering said contrast agent to a patient, and scanning the patient using diagnostic imaging. More particularly, the present invention provides a contrast agent comprising either a liquid perfluorocarbon or a gaseous perfluorocarbon encapsulated by a composition of tile formula A-B, wherein A is a lipid or lipophilic moiety and B is a negatively charged component, with the provisos that the contrast agent has an overall negative charge, and that when the contrast agent includes a gaseous perfluorocarbon, the negatively charged component B is at least one of carboxylic acid, tetrazole, boronic acid, phosphonic acid, phosphinic acid, sulfonic acid, or a compound of the Formula I: 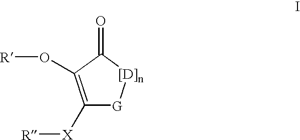
wherein:

n is 0, 1, 2, or 3;

D is C(═O);

G is a bond or C(OH)═C(OH);

R′ is H, a pharmaceutically acceptable cation, or a bond to said lipid or lipophilic moiety;

R′ is H, or a bond to said lipid or lipophilic moiety; and

X is O, S, NR′″, or C(R′″)₂, wherein R′″ is, independently, H or C1-C6 alkyl.

The present invention is directed to these, as well as other important ends.

### DETAILED DESCRIPTION OF ILLUSTRATIVE EMBODIMENTS

The present invention is directed, in part, to contrast agents that possesses an overall negative charge, and more particularly to a contrast agent comprising either a liquid perfluorocarbon or a gaseous perfluorocarbon encapsulated by a composition of the formula A-B, wherein A is a lipid or lipophilic moiety and B is a negatively charged component, with the provisos that the contrast agent has an overall negative charge, and that when the contrast agent includes a gaseous perfluorocarbon, the negatively charged component B is at least one of carboxylic acid, tetrazole, boronic acid, phosphonic acid, phosphinic acid, sulfonic acid, or a compound of the Formula I: 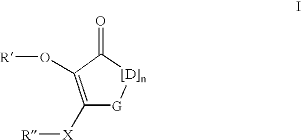
wherein:

n is 0, 1, 2, or 3;

D is C(═O);

G is a bond or C(OH)═C(OH);

R′ is H, a pharmaceutically acceptable cation, or a bond to said lipid or lipophilic moiety;

R′ is H, or a bond to said lipid or lipophilic moiety; and

X is O, S, NR′″, or C(R′″)₂, wherein R′″ is, independently, H or C1-C6 alkyl.

Examples of gas filled imaging moieties include those found in U.S. patent application Ser. No. 091931,317, filed Aug. 16, 2001, and U.S. Pat. Nos. 5,088,499, 5,547,656, 5,228,446, 5,585,112, and 5,846,517, the disclosures of which are hereby incorporated herein by reference in their entireties. Another example of perfluorocarbon lipid-encapsulated nanoparticle is disclosed in U.S. Ser. No. 60/351,390 filed Jan. 24, 2002 (WO 03/062198, published Jul. 31, 2003) the disclosure of which is incorporated herein as if reproduced in its entirety.

In one embodiment, the perfluorocarbon encapsulated by the lipid composition is an echogenic entity for use in ultrasound imaging, such as is described in U.S. Pat. No. 5,585,112, U.S. Pat. No. 5,773,024, and PCT Application No. PCT/US99/00747, filed Jan. 14, 1999 (WO99/36104), the disclosures of which are hereby incorporated by reference herein in their entireties.

In one embodiment, perfluorocarbon is perfluorodecalin, perfluorooctane, perfluorodichlorooctane, perfluoro-n-octyl bromide, perfluoroheptane, perfluorodecane, perfluorocyclohexane, perfluoromorpholine, perfluorotripropylamine, perfluortributylamine, perfluorodimethylcyclohexane, perfluorotrimethylcyclohexane, perfluorodicyclohexyl ether, perfluoro-n-butyltetrahydrofuran, and compounds that are structurally similar to these compounds and are partially or fully halogenated (including at least some fluorine substituents) or partially or fully fluorinated including perfluoroalkylated ether, polyether or crown ether. In one embodiment, the perfluorocarbon is perfluorocyclohexane, perfluorodecalin, or perfluorooctyl bromide. Preferably, the perfluorocarbon is perfluorooctyl bromide.

In one embodiment, the liquid perfluorocarbon is perfluorooctane.

In one embodiment, the gaseous perfluorocarbon is perfluoropropane.

In one embodiment, said lipophilic moiety A is C4 to C20 hydrocarbon. In another embodiment, said lipophilic moiety A is C8 to C 16 hydrocarbon. The functionality A may be linear or branched, saturated or possessing olefinic, alkynyl, aryl, or heteroaryl functionalities within or attached to the chain length.

In yet another embodiment, said lipid or lipophilic moiety A is a lipid. In one embodiment, said lipid A is dipalmitoyl phosphatidyl serine, dipalmitoyl phosphatidyl ethanolamine, or dipalmitoyl phosphatidyl N-methylethanolamine.

In one embodiment, the lipid composition further comprises an architectural lipid comprising at least one of natural or synthetic phospholipids, fatty acids, cholesterols, glycolipids, lysolipids, sphingomyelins, or lipid conjugated polyethylene glycol. The preferred lipids for the construction of the nanoparticle are dipalmitoyl phosphatidyl serine (DPPS), dipalmitoyl phosphatidic acid (DPPA), dipalmitoyl phosphatidyl ethanolamine (DPPE), and dipalmitoyl phosphatidyl choline (DPPC). In the case of DPPA and DPPC, sufficient negatively charged moieties must be incorporated to neutralize the amine and/ or overcome the positive charge from the choline such that the surface of the nanoparticle or liposome possesses an overall negative charge.

The functionality to impart a suitable net negative charge to the surface of the nanoparticle (sometimes also referred to as liposome) is selected from the group of known functionalities that formally are negatively charged, Such functionalities include carboxylic acids, tetrazoles, boronic acids, phosphonic acids, phosphinic acids, sulfonic acids, oxocarbon acid monoesters (e.g. tetradecyl squarate or tetradecyl croconate), and other suitable negatively charged functionalities. In a preferred embodiment, the negatively charged species localizes in specified tissues due to selective uptake from or affinity to said tissue.

In one embodiment, the negatively charged component B is at least one of carboxylic acid, tetrazole, boronic acid, phosphonic acid, phosphinic acid, sulfonic acid, or a compound of the Formula I: 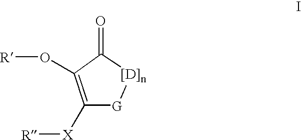
wherein:

n is 0, 1, 2, or 3;

D is C(═O);

G is a bond or C(OH)═C(OH);

R′ is H, a pharmaceutically acceptable cation, or a bond to said lipid or lipophilic moiety;

R′ is H, or a bond to said lipid or lipophilic moiety; and

X is O, S, NR′″, or C(R′″)₂, wherein R′″ is, independently, H or C1-C6 alkyl.

In one embodiment, X is O and R″ is a bond to said lipid or lipophilic moiety. The aforementioned B groups may be attached to A via an amine, amido, oxo, or ester functionality. The chemistry of these acids, and their corresponding monoesters and monoamides is well known, and the conjugation of such should be apparent to anyone ordinarily skilled in the art.

In one embodiment, the pharmaceutically acceptable cation is lithium, sodium, potassium, tetraalkylammonium, magnesium, or calcium.

Boronic acid can form a selective and covalent bond with a specific conformation of glycol (e.g. sugars). In the free acid form, the boronic acid can exist as the anionic. borate complex. In one embodiment, the boronic acid is substituted on a steric or electronically selective scaffold to impart specificity to the interaction. Such examples are boronic acid-appended metalloporphyrins (Shinkai, et al., Tetrahedron Letter's, 1995, (36), 2093-2096), bis-phenylboronic acid anthracenes (Shinkai, et al. J. Am. Chem. Soc., 1995, (117), 8982), extended polyaromatic systems (Drueckhammer, et al. Angnew. Chem. Intl. Ed Engl., 2001, (40), 1714), and dianthracene boronic acids (Wang, et al. Bioorg. Med Chem. Letter's, 2002, (12), 3373-3377). Due to the mechanism of boronic ester formation, a thermodynamically stable conformation can be selected. One such example is the selectivity of binding glucose over fructose (43-fold) as well as galactose (49-fold, Wang, 2002). In another embodiment, bis- or tris- boronic acid is structurally held in such a way as to impart selective binding via multiple boronic ester formation. These esters are formed in a thermodynamic fashion through either steric or electronic forces.

Tetrazole and phosphonate each afford an anionically charged, non-hydrogen bonding functionality.

Deltate, squarate, croconate, and rhodizonate are also anionically charged functionalities. 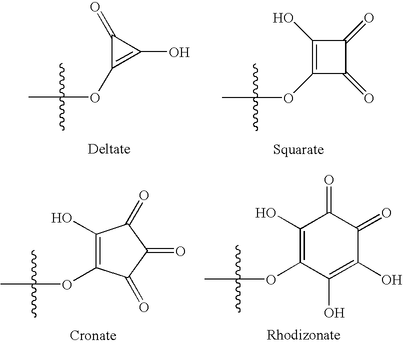
While rhodizonates afford the desired negative charge, they are less preferred due to their inherent tendency to convert spontaneously to the croconate by extrusion of a CO unit.

In one embodiment, the negatively charged component B is an oxocarbon acid monoester. In another embodiment, B is a boronic acid. In another embodiment, B is a tetrazole. In yet another embodiment, B is a phosphonic acid.

In one embodiment, said lipid or lipophilic moiety and negatively charged component are selected from:

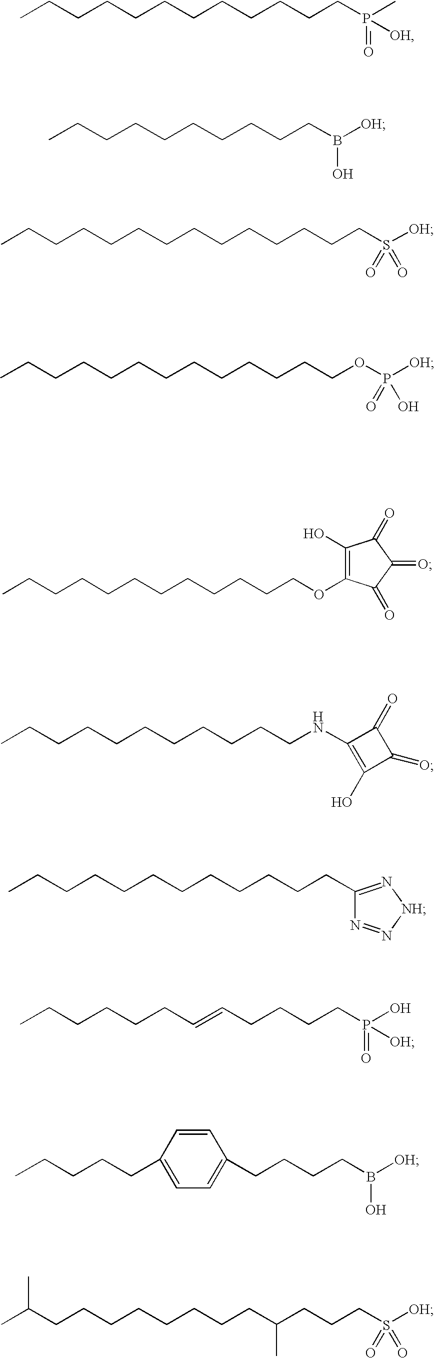

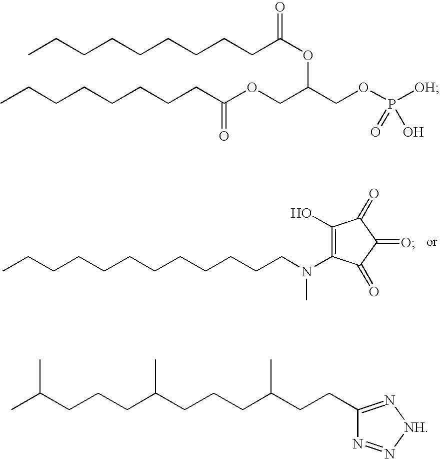

In one embodiment, a portion of the lipid composition is attached to a chelator directly or through a linking group such as polyethylene glycol (PEG).

In one embodiment, the chelator is useful for chelating a metal, such as a paramagnetic metal.

In one embodiment, the chelator has a formula selected from the group: 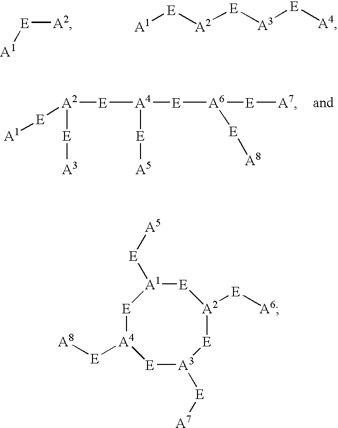
wherein:

A¹, A², A³, A⁴, A⁵, A⁶, A⁷, and A⁸ are independently selected at each occurrence from the group: NR₁, NR₁R₂, S, SH, S(Pg), O, OH, PR₁, PR₁R₂, P(O)R₃R_{4,} and a bond to L;

E is a bond, CH, or a spacer group independently selected at each occurrence from the group: C₁₋₁₀ alkyl substituted with 0-3 R₅, aryl substituted with 0-3 R₅, C₃₋₁₀ cycloalkyl substituted with 0-3 R₅, heterocyclo C₁₀₁₀ alkyl substituted with 0-3 R₅, wherein the heterocyclo group is a 5-10 membered heterocyclic ring system containing 1-4 heteroatoms independently selected from N, S, and O, C₆₋₁₀ aryl-C₁₋₁₀ alkyl substituted with 0-3 R₅, C₁₋₁₀ alkyl-C₆₋₁₀ aryl substituted with 0-3 R₅, and a 5-10 membered heterocyclic ring system containing 1-4 heteroatoms independently selected from N, S, and O and substituted with 0-3 R₅;

R₁ and R₂ are each independently selected from the group: a bond to L, hydrogen, C₁₋₁₀ alkyl substituted with 0-3 R₅, aryl substituted with 0-3 R₅, C₃₋₁₀ cycloalkyl substituted with 0-3 R₅, heterocyclo C₁₋₁₀ alkyl substituted with 0-3 R₅, wherein the heterocyclo group is a 5-10 membered heterocyclic ring system containing 1-4 heteroatoms independently selected from N, S, and O, C₆₋₁₀ aryl-C₁₋₁₀ alkyl substituted with 0-3 R₅, C₁₋₁₀ alkyl-C₆₋₁₀ aryl substituted with 0-3 R₅, and a 5-10 membered heterocyclic ring system containing 1-4 heteroatoms independently selected from N, S, and O and substituted with 0-3 R₅, and an electron, provided that when one of R₁ or R₂ is an electron, then the other is also an electron;

alternatively, R₁ and R₂ combine to form ═C(R₆)(R₇);

R₃ and R₁₄ are each independently selected from the group: a bond to L, OH, C₁₋₁₀ alkyl substituted with 0-3 R₅, aryl substituted with 0-3 R₅, C₃₋₁₀ cycloalkyl substituted with 0-3 R₅, heterocyclo C₁₋₁₀ alkyl substituted with 0-3 R₅, wherein the heterocyclo group is a 5-10 membered heterocyclic ring system containing 1-4 heteroatoms independently selected from N, S, and O, C₆₋₁₀ aryl-C₁₋₁₀ alkyl substituted with 0-3 R₅, C₁₋₁₀ alkyl-C₆₋₁₀ aryl substituted with 0-3 R₅, and a 5-10 membered heterocyclic ring system containing 1-4 heteroatoms independently selected from N, S, and O and substituted with 0-3 R₅;

R₅ is independently selected at each occurrence from the group: a bond to L, ═O, F, Cl, Br, I, CF₃, CN, CO₂R₈, C(═O)R₈, C(═O)N(R₈)₂, CHO, CH₂OR₈, OC(═O)R₈, OC(═O)OR₈ₐ, OR₈, OC(═O)N(R₈)₂, NR₉C(═O)R₈, NR₉C(═O)OR₈ₐ, NR₉C(═O)N(R₈)₂, NR₉SO₂N(R₈)₂, NR₉SO₂R₈ₐ, SO₃H, SO₂R₈ₐ, SR₈, S(═O)R₈ₐ, SO₂N(R₈)₂, N(R₈)₂, NHC(═S)NHR₈, NOR₈, NO₂, C(═O)NHOR₈, C(═O)NHNRR₈R₈ₐ, OCH₂CO₂H, 2-(1-morpholino)ethoxy, C₁₋₅ alkyl, C₂₋₄ alkenyl, C₃₋₆ cycloalkyl, C₃₋₆ cycloalkylmethyl, C₂₋₆ alkoxyalkyl, aryl substituted with 0-2 R₈, and a 5-10 membered heterocyclic ring system containing 1-4 heteroatoms independently selected from N, S, and O;

R₈, R₈ₐ, and R₉ are independently selected at each occurrence from the group: a bond to L, H, C₁₋₆ alkyl, phenyl, benzyl, C₁₋₆ alkoxy, halide, nitro, cyano, and trifluoromethyl;

Pg is a thiol protecting group;

R₆ and R₇ are independently selected from the group: H, C₁₋₁₀ alkyl, CN, CO₂R₁₀, C(═O)R₁₀, C(═O)N(R₁₀)₂, C₂₋₁₀ 1-alkene substituted with 0-3 R₁₁, C₂₋₁₀ 1-alkyne substituted with 0-3 R₁₁, aryl substituted with 0-3 R₁₁, unsaturated 5-10 membered heterocyclic ring system containing 1-4 heteroatoms independently selected from N, S, and O and substituted with 0-3 R₁₁, and unsaturated C₃₋₁₀ carbocycle substituted with 0-3 R₁₁;

alternatively, R₆ and R₇, taken together with the divalent carbon radical to which they are attached form:

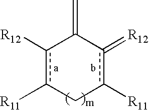

a and b indicate the positions of optional double bonds and m is 0 or 1;

R₁₁ and R₁₂ are independently selected from the group: H, R₁₃, C₁₋₁₀ alkyl substituted with 0-3 R₁₃, C₂₋₁₀ alkenyl substituted with 0-3 R₁₃, C₂₋₁₀ alkynyl substituted with 0-3 R₁₃, aryl substituted with 0-3 R₁₃, a 5-10 membered heterocyclic ring system containing 1-4 heteroatoms independently selected from N, S, and O and substituted with 0-3 R₁₃, and C₃₋₁₀ carbocycle substituted with 0-3 R₁₃;

alternatively, R₁₁ and R₁₂ taken together form a fused aromatic or a 5-10 membered heterocyclic ring system containing 1-4 heteroatoms independently selected from N, S, and O;

R₁₃ is independently selected at each occurrence from the group: O, F, Cl, Br, I, CF₃, CN, CO₂R₁₀, C(═O)R₁₀, C(═O)N(R₁₀)₂, N(R₁₀)₃₊, CH₂OR₁₀, OC(═O)R₁₀, OC(═O)OR₁₋ₐ, OR₁₀, OC(═O)N(R₁₀)₂, NR₁₄C(═O)R₁₀, NR₁₄C(═O)OR₁₀ₐ, NR₁₄C(═O)N(R₁₀)₂, NR₁₄SO₂N(R₁₀)₂, NR₁₄SO₂R₁₀ₐ, SO₃H, SO₂R₁₀ₐ, SR₁₀, S(═O)R₁₀ₐ, SO₂N(R₁₀)₂, N(R₁₀)₂, ═NOR₁₀, C(═O)NHOR₁₀, OCH₂CO₂H, and 2-(1-morpholino)ethoxy; and,

R₁₀, R₁₀ₐ, and R₁₄ are each independently selected at each occurrence from the group: hydrogen and C₁₋₆, alkyl;

or a pharmaceutically acceptable salt thereof.

In a preferred embodiment, the chelator is DOTA or DPTA.

In one embodiment, the metal is a paramagnetic species for use in MRI imaging. Preferably, the paramagnetic species for use in MRI imaging is an isotope selected from the group Gd³⁺, Fe³⁺, In³⁺, and Mn²⁺. More preferably, the paramagnetic species is chelated Gd⁺³.

In one embodiment, the present invention provides a contrast agent comprising:

a liquid perfluorocarbon encapsulated by a lipid composition, the lipid composition comprising:

i) a composition of the formula A-B, wherein A is a lipid or lipophilic moiety and B is a negatively charged component;

ii) an architectural lipid; and

iii) a composition of the formula A-Ch wherein Ch is a chelator, with the proviso that the contrast agent has an overall negative charge. It is understood the components are as described above.

In another embodiment, the present invention provides a contrast agent comprising:

a gaseous perfluorocarbon encapsulated by a lipid composition, the lipid composition comprising:

i) a composition of the formula A-B, wherein A is a lipid or lipophilic moiety and B is at least one of carboxylic acid, tetrazole, boronic acid, phosphonic acid, phosphinic acid, sulfonic acid, or a compound of the Formula I: 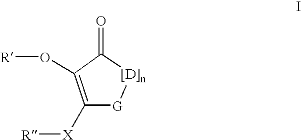
wherein:

n is 0, 1, 2, or 3;

D is C(=O);

G is a bond or C(OH)=C(OH);

R′ is H, a pharmaceutically acceptable cation, or a bond to said lipid or lipophilic moiety;

R′ is H, or a bond to said lipid or lipophilic moiety; and X is O, S, NR′″, or C(R′″)₂, wherein R′″ is, independently, H or C1-C6 alkyl;

ii) an architectural lipid; and

iii) a composition of the formula A-Ch wherein Ch is a chelator, with the proviso that the contrast agent has an overall negative charge. It is understood the components are as described above.

In yet another embodiment of the invention, a nanoparticle containing a perfluorocarbon (such as perfluorocyclohexane, perfluorodecalin, perfluorooctyl bromide) is encapsulated by a lipid mixture from the list of DPPC, DPPE, and cholesterol, with a lipid or lipophilic group bearing a chelate (e.g. DOTA or DTPA) bound to a paramagnetic metal (e.g. Mn+2 or Gd+3) and a lipid or lipophilic group bearing a negatively charged group (e.g. borate, tetrazole, phosphonate, sulfonate) at one end of the molecule. These nanoparticles are formed in the usual manner and from a mixture of the lipid-layer components and the fluorocarbon core.

In one embodiment, the contrast agent is a liposome that is formed in a fashion similarly described in Chonn et. al. (J. Immun., 1991, 146, 4234-4241). The liposomes are formed with the substitution of a long chain boronic acid (e.g. tridecylboronic acid ) for the stearyl amine component.

In another embodiment, the contrast agent is a nanoparticle containing a perfluorooctyl bromide core (20-40% v/v) encapsulated by a vegetable oil (safflower, 2% w/v), a mixture containing Gd-MeO-DOTA-PE (Hexadecanoic acid 2- {[2-(3- {3-[carboxy-(4,7, 10-tris-carboxymethyl- 1,4,7,10 tetraaza- cyclododec-1-yl)-methyl]4-methoxy-phenyl}-thioureido)-ethoxy]-hydroxy-phosphloryloxy)-1-hexadecanoyloxymethyl-ethyl ester, gadolinium (+3) salt), DPPS, cholesterol, and tetradecylphosphonic acid (ca. 2% w/v, ca. 30;30;20;20 molar proportions).

In another embodiment, the contrast agent is a nanoparticle containing a perfluorooctyl bromide core (20-40% v/v) encapsulated by a vegetable oil (safflower, 2% w/v), a mixture containing Gd-MeO-DOTA-PE, DPPS, cholesterol, and tetradecylsulphonic acid (ca. 2% w/v, ca, 30:30:20:20 molar proportions).

In another embodiment, the contrast agent is a nanoparticle containing a perfluorooctyl bromide core (20-40% v/v) encapsulated by a vegetable oil (safflower, 2% w/v), a mixture containing Cd-MeO-DOTA-PE, DPPS, cholesterol, and tetradecylboronic acid (ca. 2% w/v, ca. 30:30:20:20 molar proportions).

In another embodiment, the contrast agent is a nanoparticle containing a perfluorooctyl bromide core (20-40% v/v) encapsulated by a vegetable oil (safflower, 2% w/v), a mixture containing Gd-MeO-DOTA-PE, DPPS, cholesterol, and a bis- or tris-boronic acid compound (such as those described in Cabell, L. A.; Monahan, M.-K.; Anslyn, E. V. Tetrahedron Letters (1999), 40(44), 7753-7756; Iarnati, V. V.; Gao, X.; Gao, S.; Yang, W.; Ni, W.; Sanlkar, S.; Wang, B. Bioorg. & Med. Chem. Letters (2002), 12(23), 3373-3377, and Wang, B.; Weston, B.; Yang, W. PCT Int. Appl. WO 2003094926 A1) linked to an alkyl chain in an appropriate manner (ca. 2% w/v, ca. 30:30:20:20 molar proportions).

Additional targeting moieties may be incorporated into or attached onto the surface of the nanoparticle. In the instance in which a targeting moiety is employed, the purpose is to impart a greater selectivity to the localization of the nanoparticle to enhance the image quality.

For example, contrast agents for myocardium perfusion imaging typically comprise a targeting moiety for rendering the contrast agent tissue specific, an imaging moiety for a establishing a detectable signal, and an optional linking group. For example, myocardial reperfusion injury is characterized by undesirable angiogenesis, which depends on the agency of αvβ3 integrin (or “vitronectin receptor”). Thus, vitronectin receptor targeting moieties have been developed for therapeutic and diagnostic pharmaceuticals. See, e.g., U.S. Pat. Nos. 6,548,663, 6,524,553, 6,511,649, 6,511,648, and 6,322,770.

Linking Groups/Pharmacokinetic Modifiers

Pharmacokinetic modifiers may or may not be incorporated into or affixed onto the surface of the nanoparticle. Generally speaking, a pharmacokinetic modifier is a compound that serves to direct the biodistribution of an injected pharmaceutical, apart from the interaction of the targeting moiety with the receptor. Pharmacokinetic modifiers can be used, for example, to enhance or decrease hydrophilicity and rate of blood clearance, as well as to direct the route of elimination of the pharmaceuticals. Preferred pharmacokinetic modifiers are those that result in moderate to fast blood clearance and enhanced renal excretion. A wide variety of functional groups can serve as pharmacokinetic modifiers, including, but not limited to, carbohydrates, polyalkylene glycols, peptides or other polyamino acids, and cyclodextrins.

The pharmacokinetic modifier may be incorporated into or attached onto the surface of the nanoparticle or liposome and can be attached via an immobilization moiety such as one of the following: a phosphoryl akylamine (e.g., as in DPPA), an alkyl bond to a lipid, or lipiphilic chain (long-chain alkyl, aryl, sterol, etc.)

The attachment of the pharmacokinetic modifier may be direct via a bond, linking group, or a functionality that itself may serve as a linking group. These linkers may take the form of a polyethyleneglycol (PEG), polyalkylamine, long-chain alkyl or arylalkyl, or other suitable functionality.

In another embodiment, the linking group is as set forth in U.S. Pat. No. 6,548,663 as Lₙ, the disclosure of which is hereby incorporated herein by reference in its entirety.

In another embodiment, the linking group is as set forth in U.S. Pat. No. 6,524,553 as Lₙ, the disclosure of which is hereby incorporated herein by reference in its entirety.

In another embodiment, the linking group is as set forth in U.S. Pat. No. 6,511,649 as Lₙ, the disclosure of which is hereby incorporated herein by reference in its entirety.

In another embodiment, the linking group is as set forth in U.S. Pat. No. 6,511,648 as Lₙ, the disclosure of which is hereby incorporated herein by reference in its entirety.

In another embodiment, the linking group is as set forth in U.S. Pat. No. 6,332,770 as Lₙ, the disclosure of which is incorporated herein by reference in its entirety.

In another embodiment, the linking group is as set forth in U.S. patent application Ser. No. 09/281,474, filed Mar. 30, 1999 (DM-6958) as Lₙ, the disclosure of which is hereby incorporated herein by reference in its entirety.

Methods

In one embodiment of the present invention, a method of imaging myocardium perfusion is provided, comprising administering the contrast agents described herein to a patient and scanning the patient using diagnostic imaging.

In one embodiment, the imaging comprises at least one of MRI imaging or ultrasound imaging.

Methods Of Making

In one embodiment, contrast agents can be prepared by blending the desired lipids (A-B, architectural lipids, and A-Ch (if present)) in methanol and chloroform, then removing the solvent to form a homogenous solid. Then about 2% wt/vol of the blend is combined with about 200 mL (vol/vol) perfluorocarbon and sufficient water to make one liter, This mixture is emulsified by conventional methods.

Use

The contrast agents of the present invention may be used in a method of imaging, including methods of imaging in a patient comprising administering the contrast agent to the patient by injection, infusion, or any other known method, and imaging the area of the patient wherein the event of interest is located.

The useful dosage to be administered and the particular mode of administration will vary depending upon such factors as age, weight, and particular region to be treated, as well as the particular contrast agent used, the diagnostic use contemplated, and the form of the formulation, for example, suspension, emulsion, microsphere, liposome, or the like, as will be readily apparent to those skilled in the art.

Typically, dosage is administered at lower levels and increased until the desirable diagnostic effect is achieved. In one embodiment, the above-described contrast agents may be administered by intravenous injection, usually in saline solution, at a dose of about 0.1 to about 100 mCi per 70 kg body weight (and all combinations and subcombinations of dosage ranges and specific dosages therein), or preferably at a dose of about 0.5 to about 50 mCi. Imaging is performed using techniques well known to the ordinarily skilled artisan. For example, the ultrasound contrast agents of the present invention are administered by intravenous injection in an amount of 10 to 30 μL of the echogenic gas per kg body weight or by infusion at a rate of approximately 3 μL/kg/min. Imaging is performed using known techniques of sonography.

For use as nuclear medicine contrast agents, the compositions of the present invention, dosages, administered by intravenous injection, will typically range from about 0.5 μmol/kg to about 1.5 mmol/kg (and all combinations and subcombinations of dosage ranges and specific dosages therein), preferably about 0.8 μmol/kg to about 1.2 mmol/kg.

For use as MRI contrast agents, the compositions of the present invention may be used in a similar manner as other MRI agents as described in U.S. Pat. No. 5,155,215; U.S. Pat. No. 5,087,440; Margerstadt et al., Magn. Reson. Med., 1986, 3, 808; Range et al., Radiology, 1988, 166, 835; and Bousquet et al., Radiology, 1988, 166, 693. The disclosures of each of the foregoing documents are hereby incorporated herein by reference in their entireties. Generally, sterile aqueous solutions of the contrast agents may be administered to a patient intravenously in dosages ranging from about 0.01 to about 1.0 mmoles per kg body weight (and all combinations and subcombinations of dosage ranges and specific dosages therein).

The ultrasound contrast agents of the present invention may be administered by intravenous injection in an amount from about 10 to about 30 μL (and all combinations and subcombinations of dosage ranges and specific dosages therein) of the echogenic gas per kg body weight or by infusion at a rate of approximately 3 μL/kg/min.

Buffers useful in the preparation of contrast agents and kits include, for example, phosphate, citrate, sulfosalicylate, and acetate buffers. A more complete list can be found in the United States Pharmacopoeia, the disclosure of which is hereby incorporated herein by reference, in its entirety.

Lyophilization aids useful in the preparation of contrast agents and kits include, for example, mannitol, lactose, sorbitol, dextran, FICOLL® polymer, and polyvinylpyrrolidine (PVP).

Stabilization aids useful in the preparation of contrast agents and kits include, for example, ascorbic acid, cysteine, monothioglycerol, sodium bisulfite, sodium metabisulfite, gentisic acid, and inositol.

Solubilization aids useful in the preparation of contrast agents and kits include, for example, ethanol, glycerin, polyethylene glycol, propylene glycol, polyoxyethylene sorbitan monooleate, sorbitan monoloeate, polysorbates, poly(oxyethlylene)-poly(oxypropylene)-poly(oxyethylene) block copolymers (“Pluronics”) and lecithin. Preferred solubiliziing aids are polyethylene glycol and Pluronics.

Bacteriostats useful in the preparation of contrast agents and kits include, for example, benzyl alcohol, benzalkonium chloride, chlorbutanol, and methyl, propyl, or butyl paraben.

A component in a diagnostic kit can also serve more than one function. For example, a reducing agent for a radionuclide can also serve as a stabilization aid, or a buffer can also serve as a transfer ligand, or a lyophilization aid can also serve as a transfer, ancillary, or co-ligand.

The compounds herein described may have asymmetric centers. Unless otherwise indicated, all chiral, diastereomeric and racemic forms are included in the present invention. Many geometric isomers of olefins, C═N double bonds, and the like can also be present in the compounds described herein, and all such stable isomers are contemplated in the present invention. It will be appreciated that compounds of the present invention contain asymmetrically substituted carbon atoms, and may be isolated in optically active or racemic forms. It is well known in the art how to prepare optically active forms, such as by resolution of racemic forms or by synthesis from optically active starting materials. Two distinct isomers (cis and trans) of the peptide bond are known to occur; both can also be present in the compounds described herein, and all such stable isomers are contemplated in the present invention The D and L-isomers of a particular amino acid are designated herein using the conventional 3-letter abbreviation of the amino acid, as indicated by the following examples: D-Leu, or L-Leu.

When any variable occurs more than one time in any substitutent or in any formula, its definition in each occurrence is independent of its definition at every other occurrence. Thus, for example, if a group, or plurality of groups, is shown to be substituted with 0-2 R⁵², then said group(s) may optionally be substituted with up to two R⁵², and R⁵² at each occurrence in each group is selected independently from the defined list of possible R⁵². Also, by way of example, for the group —N(R⁵³)₂, each of the two R⁵³ substituents on N is independently selected from the defined list of possible R⁵³. Combinations of substituents and/or variables are permissible only if such combinations result in stable compounds. For example, when n is 3 in Formula I, G is a bond.

When a bond to a substituent is shown to cross the bond connecting two atoms in a ring, then such substituent may be bonded to any atom on the ring.

Definitions

As used herein, “alkyl” is intended to include both branched and straight-chain saturated aliphatic hydrocarbon groups having the specified number of carbon atoms, examples of which include, but are not limited to, methyl, ethyl, n-propyl, i-propyl, n-butyl, i-butyl, sec-butyl, t-butyl, pentyl, hexyl, heptyl, octyl, nonyl, and decyl; cycloalkyl including saturated and partially unsaturated ring groups, including mono-, bi- or poly-cyclic ring systems, such as cyclopropyl, cyclobutyl, cyclopentyl, cyclohexyl, cycloheptyl, cyclooctyl and adamantyl; bicycloalkyl including saturated bicyclic ring groups such as [3.3.0]bicyclooctane, [4.3.0]bicyclononane, [4.4.0]bicyclodecane (decalin), [2.2.2]bicyclooctane, and so forth.

The term “alkoxy” means an alkyl-CO- group wherein alkyl is as previously described. Exemplary groups include methoxy, ethoxy, and so forth.

As used herein, the term “alkyne” or “alkenyl” is intended to include hydrocarbon chains having the specified number of carbon atoms of either a straight or branched configuration and one or more unsaturated carbon-carbon bonds which may occur in any stable point along the chain, such as ethenyl, propenyl, and the like.

As used herein, the term “alkyne” or “alkynyl” is intended to include hydrocarbon chains having the specified number of carbon atoms of either a straight or branched configuration and one or more unsaturated carbon-carbon triple bonds which may occur in any stable point along the chain, such as propargyl, and the like.

As used herein, the term alkyl ether is intended to include hydrocarbon chains having the specified number of carbon atoms of either a straight or branched configuration and a noncarbon atom, such as S or O, with two points of attachment (i.e., is a diradical) in the chain.

As used herein, “aryl” or “aromatic residue” is intended to mean phenyl or naphthyl, which when substituted, the substitution can be at any position.

The term “aryloxy” means an aryl-CO- group wherein aryl is as previously described. Exemplary groups include phenoxy and naphthoxy.

As used herein, the term “alkaryl” means an aryl group bearing an alkyl group of 1-10 carbon atoms; the term “aralkyl” means an alkyl group of 1-10 carbon atoms bearing an aryl group; the term “arylalkaryl” means an aryl group bearing an alkyl group of 1-10 carbon atoms bearing an aryl group; the term “heteroaralkyl” means an alkyl group of 1-10 carbon atoms bearing an aryl group and a heteroatom; and the term “heterocycloalkyl” means an alkyl group of 1-10 carbon atoms bearing a heterocycle.

“Ancillary” or “co-ligands” are ligands that may be incorporated into a radiopharmaceutical during its synthesis. They may serve to complete the coordination sphere of the radionuclide together with the chelator or radionuclide bonding unit of the reagent. For radiopharmaceuticals comprised of a binary ligand system, the radionuclide coordination sphere may be composed of one or more chelators or bonding units from one or more reagents and one or more ancillary or co-ligands, provided that there are a total of two types of ligands, chelators or bonding units. For example, a radiopharmaceutical comprised of one chelator or bonding unit from one reagent and two of the same ancillary or co-ligands and a radiopharmaceutical comprised of two chelators or bonding units from one or two reagents and one ancillary or co-ligand are both considered to be comprised of binary ligand systems. For radiopharmaceuticals comprised of a ternary ligand system, the radionuclide coordination sphere may be composed of one or more chelators or bonding units from one or more reagents and one or more of two different types of ancillary or co-ligands, provided that there are a total of three types of ligands, chelators or bonding units. For example, a radiopharmaceutical comprised of one chelator or bonding unit from one reagent and two different ancillary or co-ligands is considered to be comprised of a ternary ligand system. Ancillary or co-ligands useful in the preparation of radiopharmaceuticals and in diagnostic kits useful for the preparation of said radiopharmaceuticals may be comprised of one or more oxygen, nitrogen, carbon, sulfur, phosphorus, arsenic, selenium, and tellurium donor atoms. A ligand can be a transfer ligand in the synthesis of a radiopharmaceutical and also serve as an ancillary or co-ligand in another radiopharmaceutical. Whether a ligand is termed a transfer or ancillary or co-ligand depends on whether the ligand remains in the radionuclide coordination sphere in the radiopharmaceutical, which is determined by the coordination chemistry of the radionuclide and the chelator or bonding unit of the reagent or reagents.

“bacteriostat” is a component that inhibits the growth of bacteria in a formulation either during its storage before use of after a diagnostic kit is used to synthesize a radiopharmaceutical.

The term “bond”, as used herein, means either a single or double bond.

The term “bubbles” or “microbubbles,” as used herein, refers to vesicles which are generally characterized by the presence of one or more membranes or walls surrounding an internal void that is filled with a gas or precursor thereto. Exemplary bubbles or microbubbles include, for example, liposomes, micelles and the like.

A “carbohydrate” is a polyhydroxy aldehyde, ketone, alcohol or acid, or derivatives thereof, including polymers thereof having polymeric linkages of the acetal type.

A “chelator” or “bonding unit” is the moiety or group on a reagent that binds to a metal ion through the formation of chemical bonds with one or more donor atoms. Preferred chelators of the present invention are described in U.S. Pat. No. 6,511,648, the disclosure of which is hereby incorporated herein by reference in its entirety,

A “cyclodextrin” is a cyclic oligosaccharide. Examples of cyclodextrins include, but are not limited to, α-cyclodextrin, hydroxyethyl-α-cyclodextrin, hydroxypropyl-α-cyclodextrin, β-cyclodextrin, hydroxypropyl-β-cyclodextrin, carboxymethyl-β-cyclodextrin, dihydroxypropyl-β-cyclodextrin, hydroxyethyl-β-cyclodextrin, 2,6 di-O-methyl-β-cyclodextrin, sulfated-β-cyclodextrin, γ-cyclodextrin, hydroxypropyl-γ-cyclodextrin, dihydroxypropyl-γ-cyclodextrin, hydroxyethyl-γ-cyclodextrin, and sulfated γ-cyclodextrin.

A “diagnostic kit” or “kit” comprises a collection of components, termed the formulation, in one or more vials which are used by the practicing end user in a clinical or pharmacy setting to synthesize diagnostic radiopharmaceuticals. The kit preferably provides all the requisite components to synthesize and use the diagnostic pharmaceutical except those that are commonly available to the practicing end user, such as water or saline for injection, a solution of the radionuclide, equipment for heating the kit during the synthesis of the radiopharmaceutical, if required, equipment necessary for administering the radiopharmaceutical to the patient such as syringes, shielding, imaging equipment, and the like. Contrast agents are provided to the end user in their final form in a formulation contained typically in one vial, as either a lyophilized solid or an aqueous solution. The end user typically reconstitutes the lyophilized material with water or saline and withdraws the patient dose or just withdraws the dose from the aqueous solution formulation as provided.

The term “donor atom” refers to the atom directly attached to a metal by a chemical bond.

The suffix “ene” when used with the hydrocarbons defined above, indicates that the group has two points of attachment (i.e., is a diradical). For example, a saturated aliphatic hydrocarbon group disposed between two other moieties would be referred to herein as “alkylene.”

As used herein, the term “heterocycle” or “heterocyclic system” is intended to mean a stable 5- to 7- membered monocyclic or bicyclic or 7- to 10-membered bicyclic heterocyclic ring which is saturated, partially unsaturated, or unsaturated (aromatic), and which consists of carbon atoms and from 1 to 4 heteroatoms independently selected from the group containing N, O and S and including any bicyclic group in which any of the above-defined heterocyclic rings is fused to a benzene ring. The nitrogen and sulfur heteroatoms may optionally be oxidized. The heterocyclic ring may be attached to its pendant group at any heteroatom or carbon atom which results in a stable structure. The heterocyclic rings described herein may be substituted on carbon or on a nitrogen atom if the resulting compound is stable. If specifically noted, a nitrogen in the heterocycle may optionally be quaternized. It is preferred that when the total number of S and O atoms in the heterocycle exceeds 1, then these heteroatoms are not adjacent to one another. It is preferred that the total number of S and O atoms in the heterocycle is not more than 1. As used herein, the term “aromatic heterocyclic system” or “heteroaryl” is intended to mean a stable 5- to 7- membered monocyclic or bicyclic or 7- to 10-membered bicyclic heterocyclic aromatic ring which consists of carbon atoms and from 1 to 4 heteroatoms independently selected from the group containing N, O and S. It is preferred that the total number of S and O atoms in the aromatic heterocycle is not more than 1. Examples of heterocycles include, but are not limited to, 1H-indazole, 2-pyrrolidonyl, 2H,6H-1,5,2-dithiazinyl, 2H-pyrrolyl, 3H-indolyl, 4-piperidonyl, 4aH1-carbazole, 4H-quinolizinyl, 6H-1,2,5-thiadiazinyl, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazalonyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b tetrahydrofuran, furanyl, furazanyl, imidazolidinyl, imidazolinyl, imidazolyl, 1H-indazolyl, indolenyl, indolinyl, indolizinyl, indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, ,1,2,4-oxadiazolyl, 1,2,5-oxadiazolyl, 1,3,4-oxadiazolyl, oxazolidinyl, oxazolyl, oxazolidinylperimidinyl, phenanthridinyl, phenanthrolinyl, phenarsazinyl, phenazinyl, phenothiazinyl, phenoxathiinyl, phenoxazinyl, phthalazinyl, piperazinyl, piperidinyl, pteridinyl, piperidonyl, 4-piperidonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, 4H-quinolizinyl, quinoxalinyl, quinuclidinyl, carbolinyl, tetrahydrofuranyl, tetrahydroisoquinolinyl, tetrahydroquiniolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, xanthenyl. Preferred heterocycles include, but are not limited to, pyridinyl, furanyl, thienyl, pyrrolyl, pyrazolyl, imidazolyl, indolyl, benzimidazolyl, 1H-indazolyl, oxazolidinyl, benzotriazolyl, benzisoxazolyl, oxindolyl, benzoxazolinyl, or isatinoyl. Also included are fused ring and spiro compounds containing, for example, the above heterocycles.

As used herein, the term “lipid” refers to a synthetic or naturally-occurring amphipathic compound which comprises a hydrophilic component and a hydrophobic component. Lipids include, for example, fatty acids, neutral fats, phosphatides, glycolipids, aliphatic alcohols and waxes, terpenes and steroids. Exemplary compositions which comprise a lipid compound include suspensions, emulsions and vesicular compositions.

“Liposome” refers to a generally spherical cluster or aggregate of amphipathic compounds, including lipid compounds, typically in the form of one or more concentric layers, for example, bilayers. They may also be referred to herein as lipid vesicles.

A “lyophilization aid” is a component that has favorable physical properties for lyophilization, such as the glass transition temperature, and is generally added to the formulation to improve the physical properties of the combination of all the components of the formulation for lyophilization.

“Metallopharmaceutical” means a pharmaceutical comprising a metal. The metal is the cause of the imageable signal in diagnostic applications. Radiopharmaceuticals are metallopharmaceuticals in which the metal is a radioisotope.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds modified by making acid or base salts. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of tile parent compound formed, for example, from non-toxic inorganic or organic acids. For example, such conventional non-toxic salts include those derived from inorganic acids such as hydrochloric, hydrobromic, sulfuric, sulfamiic, phosphoric, nitric and the like; and the salts prepared from organic acids such as acetic, propionic, succinic, glycolic, stearic, lactic, tartaric, citric, ascorbic, pamoic, maleic, hydroxymaleic, phenylacetic, glutamic, benzoic, salicylic, sulfanilic, 2-acetoxybenzoic, fumaric, toluenesufonic, methanesulfonic, ethane disulfonic, oxalic, isethionic, and the like. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, PA, 1985, p. 1418, the disclosure of which is hereby incorporated by reference.

A “polyalkylene glycol” is a polyethylene glycol, polypropylene glycol, polybutylene glycol, or similar glycol having a molecular weight of less than about 5000, terminating in either a hydroxy or alkyl ether moiety.

As used herein, the term “polycarboxyalkyl” means an alkyl group having from about two and about 100 carbon atoms and a plurality of carboxyl substituents; and the term “polyazaalkyl” means a linear or branched alkyl group having from about two and about 100 carbon atoms, interrupted by or substituted with a plurality of amine groups.

By “reagent” is meant a compound of this invention capable of direct transformation into a metallopharmaceutical of this invention. Reagents may be utilized directly for the preparation of the metallopharmaceuticals of this invention or may be a component in a kit of this invention.

A “reducing agent” is a compound that reacts with a radionuclide, which is typically obtained as a relatively unreactive, high oxidation state compound, to lower its oxidation state by transferring electron(s) to the radionuclide, thereby making it more reactive. Reducing agents useful in the preparation of radiopharmaceuticals and in diagnostic kits useful for the preparation of said radiopharmaceuticals include, for example, stannous chloride, stannous fluoride, formamidine sulfinic acid, ascorbic acid, cysteine, phosphines, and cuprous or ferrous salts. Other reducing agents are described, for example, in Brodack et. al., PCT Application 94/22496, the disclosure of which is incorporated herein by reference in its entirety.

The term “salt”, as used herein, is used as defined in the CRC Handbook of Chemistry and Physics, 65th Edition, CRC Press, Boca Raton, Fla., 1984, as any substance which yields ions, other than hydrogen or hydroxyl ions.

A “stabilization aid” is a component that is typically added to the metallopharmaceutical or to the diagnostic kit either to stabilize the metallopharmaceutical or to prolong the shelf-life of the kit before it must be used. Stabilization aids can be antioxidants, reducing agents or radical scavengers and can provide improved stability by reacting preferentially with species that degrade other components or the metallopharmaceutical.

By “stable compound” or “stable structure” is meant herein a compound that is sufficiently robust to survive isolation to a useful degree of purity from a reaction mixture, and formulation into an efficacious pharmaceutical agent.

“solubilization aid” is a component that improves the solubility of one or more other components in the medium required for the formulation.

The term “substituted”, as used herein, means that one or more hydrogens on the designated atom or group is replaced with a selection from the indicated group, provided that the designated atom's or group's normal valency is not exceeded, and that the substitution results in a stable compound. When a substituent is keto (i.e., ═O), then 2 hydrogens on the atom are replaced.

“transfer ligand” is a ligand that forms an intermediate complex with a metal ion that is stable enough to prevent unwanted side-reactions but labile enough to be converted to a metallopharmaceutical. The formation of the intermediate complex is kinetically favored while the formation of the metallopharmaceutical is thermodynamically favored. Transfer ligands useful in the preparation of metallopharmaceuticals and in diagnostic kits useful for the preparation of diagnostic radiopharmaceuticals include, for example, gluconate, glucoheptonate, mannitol, glucarate, N,N,N′,N′-ethylenediaminetetraacetic acid, pyrophosphate and methylenediphosphoniate. In general, transfer ligands are comprised of oxygen or nitrogen donor atoms.

As used herein, the term “vesicle” refers to a spherical entity which is characterized by the presence of an internal void. Preferred vesicles are formulated from lipids, including the various lipids described herein. In any given vesicle, the lipids may be in the form of a monolayer or bilayer, and the mono- or bilayer lipids may be used to form one of more mono- or bilayers. In the case of more than one mono- or bilayer, the mono- or bilayers are generally concentric. The lipid vesicles described herein include such entities commonly referred to as liposomes, micelles, bubbles, microbubbles, microspheres and the like. Thus, the lipids may be used to form a unilamellar vesicle (comprised of one monolayer or bilayer), an oligolamellar vesicle (comprised of about two or about three monolayers or bilayers) or a multilamellar vesicle (comprised of more than about three monolayers or bilayers). The internal void of the vesicles may be filled with a liquid, including, for example, an aqueous liquid, a gas, a gaseous precursor, and/or a solid or solute material, including, for example, a bioactive agent, as desired.

As used herein, the term “vesicular composition” refers to a composition which is formulate from lipids and which comprises vesicles.

As used herein, the term “vesicle formulation” refers to a composition which comprises vesicles and a bioactive agent.

The following abbreviations are used herein when present:
Acm acetamidomethyl
b-Ala, beta-Ala or bAla 3-aminopropionic acid
ATA 2-aminothiazole-5-acetic acid or 2-aminothiazole-5-acetyl group
Boc t-butyloxycarbonyl
CBZ, Cbz or Z Carbobenzyloxy
Cit citrulline
Dap 2,3-diaminopropionic acid
DCC dicyclolhexylcarbodimide
DIEA diisopropylethylamine
DMAP 4-dimethylaminiopyridine
DPPA dipalmitoyl phosphatidyl amine
DPPC dipalmitoyl phosphlatidyl choline
DPPE dipalmitoyl phosphatidyl ethanolamine
DPPS dipalmiitoyl phosphatidyl serine
EOE ethoxyethyl
HBTU 2-(l H-Benzotriazol-1-yl)-1,1,3,3-tetramethyluronium hexafluorophosphate
hynic boc-hydrazinonicotinyl group or 2-[[[5-[carbonyl]-2-pyridinyl]hydrazono]methyl]-benzenesulfonic acid,
NMeArg or MeArg a-N-methyl arginine
NMeAsp a-N-methyl aspartic acid
NMM N-methylmorpholine
OcHex O-cyclohexyl
OBzl O-benzyl
oSu O-succinimidyl
TBTU 2-(l H-Benzotriazol-1-yl)-1,1,3,3-tetramethyluronium tetrafluoroborate
THF tetrahydrofuranyl
THP tetrahydropyranyl
Tos tosyl
Tr or Trt trityl

The present invention is further described in the following examples.

### EXAMPLES

### Example 1

A mixture of Gd-MeO-DOTA-PE, DPPS, Cholesterol and tetradecylboronic acid (30:30:20:20 mole equivalents) is dissolved in chloroform-ethanol (4:1) and the solvent is removed in vaeuo. A portion of the solid (equivalent to 2% w/v) is emulsified with perfluorooctyl bromide (20% v/v) in water using a Microfludics S110 unit. The anionic nanoparticles could be terminally sterilized.

### Examples 2

A mixture of Gd-MeO-DOTA-PE, DPPA, Cholesterol and tetradecylphosphonic acid (30:30:20:50 mole equivalents) is dissolved in chloroform-ethanol (4:1) and the solvent is removed in vacuo. A portion of the solid (equivalent to 2% w/v) is emulsified with perfluorooctyl bromide (20% v/v) in water using a Microfluidics S110 unit. The anionic nanoparticles could be terminally sterilized.

### Examples 3

A mixture of(d-MeO-DOTA-PE, DPPS, Cholesterol and 2-tetradecyl-5,10-anthracen-(bis(methylaminomethyl-4-benzylboronic acid)) (30:30:20:20 mole equivalents) is dissolved in chloroform-ethanol (4:1) and the solvent is removed in vacuo. A portion of the solid (equivalent to 2% w/v) is emulsified with perfluorooctyl bromide (20% v/v) in water using a Microfluidics S110 unit. The anionic nanoparticles could be terminally sterilized.

### Example 4

A mixture of Gd-MeO-DOTA-PE, DPPC, and tetradecylsquarate monoester (3-hydroxy-4-tetradecyloxy-cyclobut-3-ene-1,2-dione) (30:30:50 mole equivalents) is dissolved in chloroform-ethanol (4:1) and the solvent is removed in vacuo. A portion of the solid (equivalent to 2% w/v) is emulsified with perfluorooctyl bromide (20% v/v) in water using a Microfluidics S110 unit. The anionic nanoparticles could be terminally sterilized.

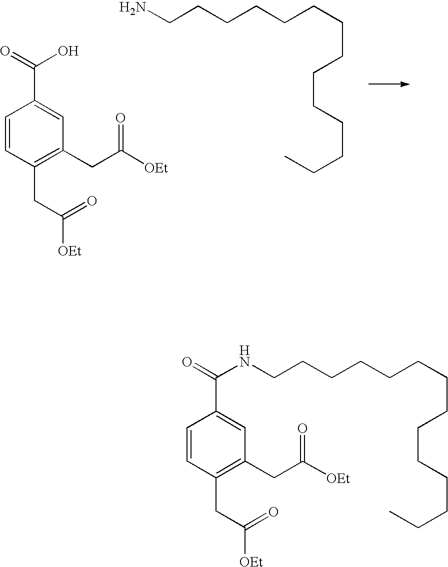

Part A: To a stirred solution of α,α′-diethyl 4-carboxy-1,2-benzenediacetate, (2.94 g, 10 mmole) and tridecylamine (1.99 g, 10 mmol) in dichloromethane (100 mL) is added HOBt (1.49 g, 11 mmol), DMAP (122 mg, 1.0 mmol), and diisopropylethylamine (1.42 g, 11 mmol). The mixture is cooled at 0 degrees C. while DCC (2.26 g, 11 mmol) is added. The mixture is stirred at 0 degrees C. for 30 minutes, the bath is removed and the mixture is stirred for two hours at room temperature. The mixture is filtered to removed DCU, and the precipitate is washed once with dichloromethane (100 mL). The combined filtrates are washed (1×200 mL 5% citric acid, 2×200 mL sat'd. aq. Na₂CO₃), dried (sat'd aq. NaCl, Na₂SO₄), and concentrated to afford a residue which is purified via column chromatography (silca gel 230-400 mesh), eluting with EtOAc-Hexanes (gradient 0-30%). Concentration of the appropriate fractions affords the desired (2-Ethoxycarbonylmethyl-4-tetradecylcarbamoyl-phenyl)-acetic acid ethyl ester.

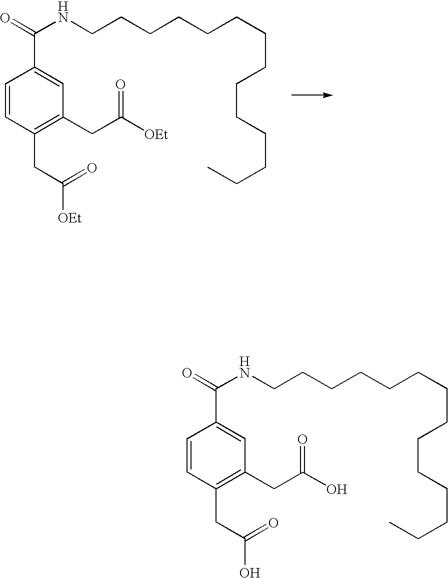

Part B: A solution of (2-Ethioxycarbonylmethyl-4-tetradecylcarbamoyl-phenyl)-acetic acid ethyl ester (2.48 g, 5 mmole) in 10% aqueous ethanol (50 mL) is stirred at room temperature while a solution of sodium hydroxide (21 mL of a 1M solution) is added. The mixture is gently heated to complete hydrolysis of both ester functionalities. The mixture is neutralized with 5M HCl, and the ethanol is removed in vacuo. Sufficient sodium chloride is added to saturate the mixture, and the resulting solution is extracted with dichloromethane (3×100 mL). The combined organics are dried (sat'd aq. NaCl, Na₂SO₄), and concentrated to afford a residue which is purified via column chromatography (silca gel 230-400 mesh), eluting with EtOAc-Hexanes (gradient I 0-50%). Concentration of the appropriate fractions affords the desired (2-Carboxymethyl-4-tetradecylcarbamoyl-phenyl)-acetic acid.

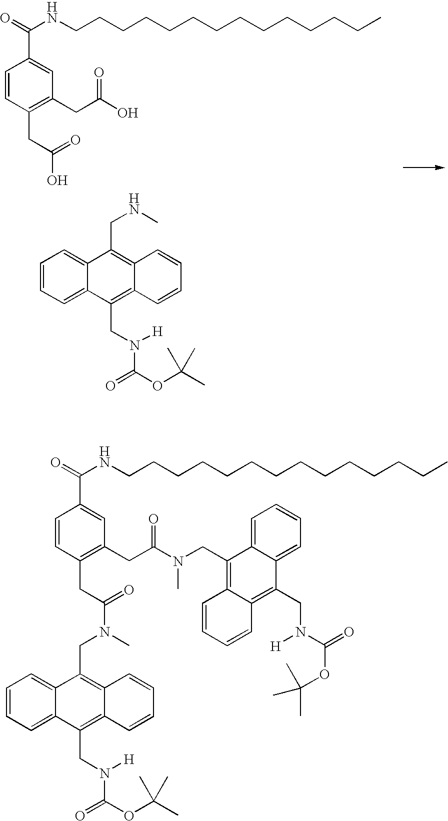

Part C: To a stirred solution of 1,1-dimethylethyl methyl[[10-[(methylamino)methyl]-9-anthracenyl]methyl]carbamate (Yang, et al. Bioorg. Med. Chem. 2002, (12), 2175; 182.2 mg, 0.5 mmol) and (2-Carboxymethyl-4-tetradecylcarbamoyl-phenyl)-acetic acid (217 mg, 0.5 mmol) in dichloromethane (20 mL) is added HOBt (73 mg, 0.55 mmol), DMAP (12 mg, 0.1 mmol), and diisopropylethylamine (71 mg, 0.55 mmol). The mixture is cooled at 0 degrees C. while EDCI (105 mg, 0.55 mmol) is added. The mixture is stirred at 0 degrees C. for 30 minutes, the bath is removed and the mixture is stirred for two hours at room temperature. Dichloromethane is added and the mixture is washed (1×200 mL 5% citric acid, 2×200 mL sat'd. aq. Na₂CO₃), dried (sat'd aq. NaCl, Na₂SO₄), and concentrated to afford a residue which is purified via column chromatography (silca gel 230-400 mesh), eluting with methanol-dichloromethane (gradient 0-1%). Concentration of the appropriate fractions affords the desired bis-anthracenyl adduct: 10-[({2-[2-({[10-(tert-butoxycarbonylamino-methyl)-anthracen-9-ylmethyl]-methyl-carbamoyl}-methyl)-5-tetradecylcarbamoyl-phenyl]-acetyl}-methyl-amino)-methyl]-anthracen-9-ylmethyl}carbamic acid tert-butyl ester.

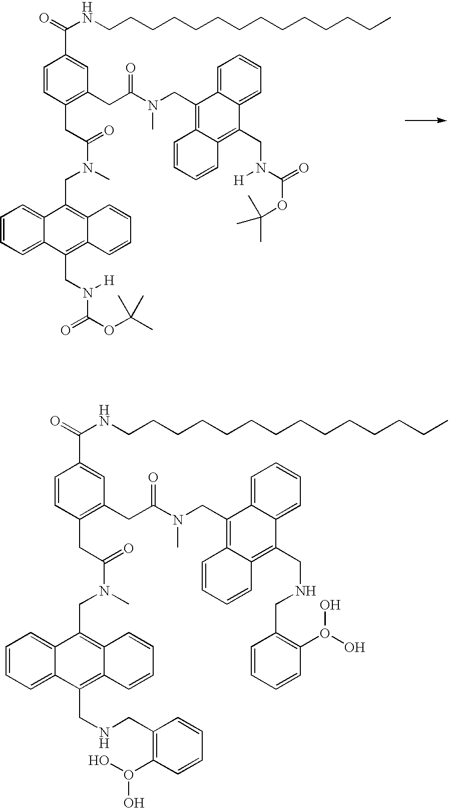

Part D: The compound prepared in Part C (219 mg, 0.2 mmol) is dissolved in dry dichloromethane (20 mL) and trifluoroacetic acid (8 mL) is added. The reaction is stirred at room temperature for 30 minutes and concentrated in vacuo. The residue is dissolved in acetonitrile (60 mL), and to the resulting solution is added K₂CO₃ (168 mg, 1.2 mmol), 2-(2-bromomethyl-phenyl)-5,5-dimethyl-{1,3,2}dioxaborinane (228 mg, 0.8 mmol), and KI (10 g). The mixture is stirred at room temperature for 12 hours, then concentrated in vacuo. The residue is dissolved in dichloromethane (80 mL) and aqueous NaHCO₃ (5%, 50 mL) is added. The resultant mixture is stirred for two hours at room temperature, the organic phase is separated, washed (3×50 mL water), dried (sat'd aq. NaCl, Na₂SO₄), and concentrated to afford a residue. This residue is purified by dissolution in dichloromethane and precipitation with hexanes.

Part E: A mixture of Gd-MeO-DOTA-PE, DPPS, Cholesterol and [5-tridecylcarboxamido-1,2-phenylenebis (1-oxo-2,1-ethanediyl)(methlylimino)methylene-10,9-anthracenediylmethylene (methylimino)methylene-2,1-phenylene]]bis-boronic acid (30:30:20:20 mole equivalents) is dissolved in chloroform-ethanol (4:1) and the solvent is removed in vacuo. A portion of the solid (equivalent to 2% w/v) is emulsified with perfluorooctyl bromide (20% v/v) in water using a Microfluidics S110 unit. The anionic nanoparticles could be terminally sterilized.

The disclosures of each patent, patent application, and publication cited or described in this document are hereby incorporated herein by reference, in their entireties.

Various modifications of the invention, in addition to those described herein, will be apparent to those skilled in the art from the foregoing description. Such modifications are also intended to fall within the scope of the appended claims.

What is claimed:

1. A contrast agent comprising:
either a liquid perfluorocarbon or a gaseous perfluorocarbon encapsulated by a composition of the formula A-B, wherein A is a lipid or lipophilic moiety and B is a negatively charged component,
with the provisos that the contrast agent has an overall negative charge, and that when the contrast agent includes a gaseous perfluorocarbon, the negatively charged component B is at least one of carboxylic acid, tetrazole, boronic acid, phosphonic acid, phosphinic acid, sulfonic acid, or a compound of the Formula I: 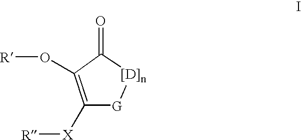
wherein:
n is 0, 1, 2, or 3;
D is C(═O);
G is a bond or C(OH)═C(OH);
R′ is H, a pharmaceutically acceptable cation, or a bond to said lipid or lipophilic moiety;
R″ is H, or a bond to said lipid or lipophilic moiety; and
X is O, S, NR′″, or C(R′″)₂, wherein R′″ is, independently, H or C1-C6 alkyl.

2. The contrast agent of claim 17 wherein said lipophilic moiety A is C4 to C20 hydrocarbon.

3. The contrast agent of claim 1, wherein said lipophilic moiety A is C8 to C16 hydrocarbon.

4. The contrast agent of claim 1, wherein said lipid or lipophilic moiety A is a lipid.

5. The contrast agent of claim 4, wherein said lipid A is dipalmitoyl phosphatidyl serine, dipalmitoyl phosphatidyl ethanolamine, or dipalmitoyl phosphatidyl N-methylethanolamine.

6. The contrast agent of claim 1, further comprising an architectural lipid comprising at least one of dipalmitoyl phosphatidyl serine (DPPS), dipalmitoyl phosphatidic acid (DPPA), dipalmitoyl phosphatidyl ethanolamine (DPPE), or dipalmitoyl phosphatidyl choline (DPPC).

7. The contrast agent of claim 1, wherein the negatively charged component B is at least one of carboxylic acid, tetrazole, boronic acid, phosphonic acid, phosphinic acid, sulfonic acid, or a compound of the Formula I: 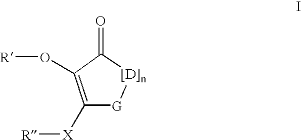
wherein:
n is 0, 1, 2, or 3;
G is a bond or C(OH)═C(OH);
R′ is H, a pharmaceutically acceptable cation, or a bond to said lipid or lipophilic moiety;
R″ is H, or a bond to said lipid or lipophilic moiety; and
X is O, S, NR′″, or C(R′″)₂, wherein R′″ is, independently, H or C1-C6 alkyl.

8. The contrast agent of claim 1, wherein X is O and R′ is a bond to said lipid or lipophilic moiety.

9. The contrast agent of claim 1, wherein the negatively charged component B is an oxocarbon acid monoester.

10. The contrast agent of claim 1, wherein the negatively charged component B is a boronic acid.

11. The contrast agent of claim 1, wherein the negatively charged component B is a tetrazole.

12. The contrast agent of claim 1, wherein the negatively charged component B is a phosphonic acid.

13. The contrast agent of claim 1, wherein said lipid or lipophilic moiety and negatively charged component are selected from:

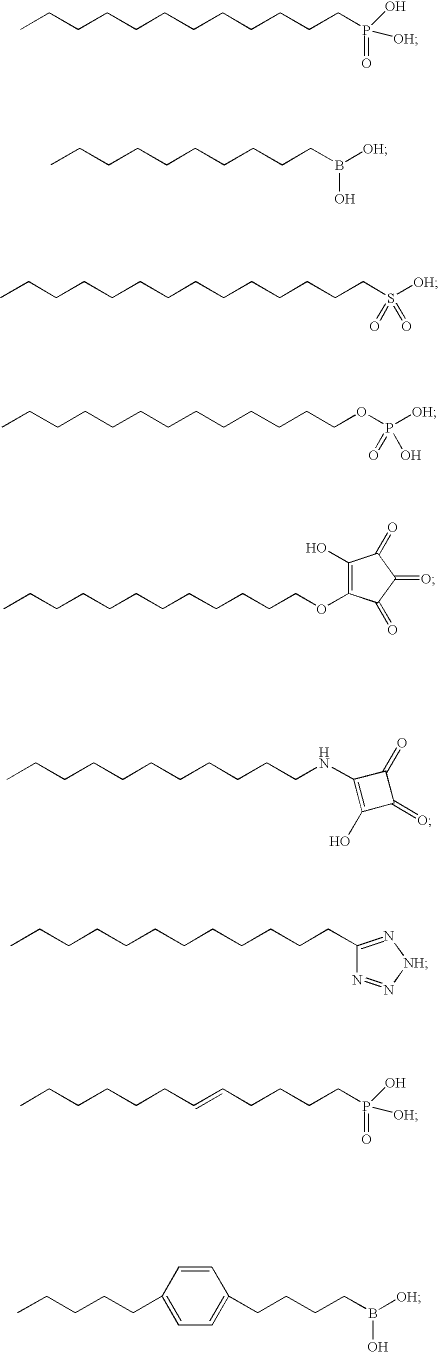

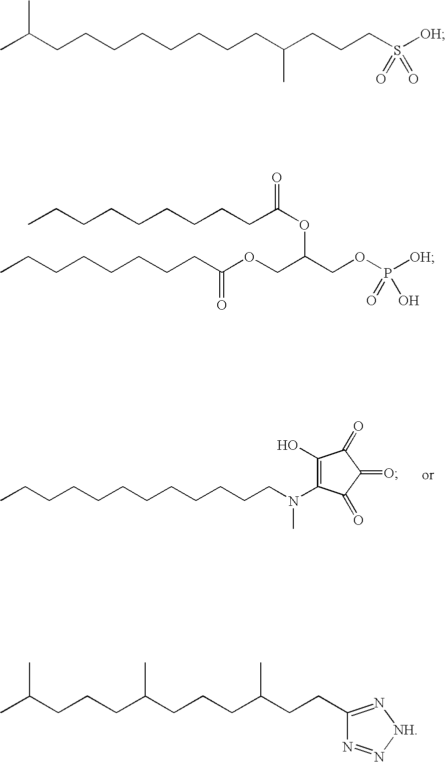

14. The contrast agent of claim 1, wherein the liquid perfluorocarbon is per fluorooctane.

15. The contrast agent of claim 1, wherein the gaseous perfluorocarbon is perfluoropropane.

16. The contrast agent of claim 6, wherein a portion of the lipid composition is attached to a chelator directly or through a linking group such as polyethylene glycol (PEG).

17. The contrast agent of claim 16, further comprising a paramagnetic species for use in MRI imaging.

18. The contrast agent of claim 17, wherein the paramagnetic species for use in MRI imaging is an isotope selected from the group Gd³⁺, Fe³⁺, In³⁺, and Mn²⁺.

19. The contrast agent of claim 16, wherein said chelator is DOTA.

20. The contrast agent of claim 16, wherein said chelator is DTPA.

21. The contrast agent of claim 16, further comprising chelated Gd⁺³.

22. The contrast agent of claim 16, wherein said chelator has a formula selected from the group: 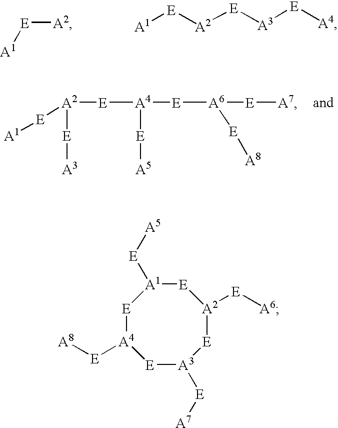
wherein:
A¹, A², A³, A⁴, A⁵, A⁶, A⁷, and A⁸ are independently selected at each occurrence from the group: NR₁, NR₁R₂, S, SH, S(Pg), O, OH, PR₁, PR₁R₂, P(O)R₃R₄, and a bond to L;
E is a bond, CH, or a spacer group independently selected at each occurrence from the group: C₁₋₁₀ alkyl substituted with 0-3 R₅, aryl substituted with 0-3 R₅, C₃₋₁₀ cycloalkyl substituted with 0-3 R₅, heterocyclo C₁₋₁₀ alkyl substituted with 0-3 R₅, wherein the heterocyclo group is a 5-10 membered heterocyclic ring system containing 1-4 heteroatom-is independently selected from N, S, and O, C₆₋₁₀ aryl-C₁₋₁₀ alkyl substituted with 0-3 R₅, C₁₋₁₀ alkyl-C₆₋₁₀ aryl substituted with 0-3 R₅, and a 5-10 membered heterocyclic ring system- containing 1-4 heteroatoms independently selected from N, S, and O and substituted with 0-3 R₅;
R₁ and R₂ are each independently selected from the group: a bond to L, hydrogen, C₁₋₁₀ alkyl substituted with 0-3 R₅, aryl substituted with 0-3 R₅, C₃₋₁₀ cycloalkyl substituted with 0-3 R₅, heterocyclo C₁₋₁₀ alkyl substituted with 0-3 R₅, wherein the heterocyclo group is a 5-10 membered heterocyclic ring system containing 1-4 heteroatoms independently selected from N, S, and O, C₆₋₁₀ aryl-C₁₋₁₀ alkyl substituted with 0-3 R₅, C₁₋₁₀ alkyl-C₆₋₁₀ aryl substituted with 0-3 R₅, and a 5-10 membered heterocyclic ring system containing 1-4 heteroatoms independently selected from N, S, and O substituted with 0-3 R₅, and an electron, provided that when one of R₁ or R₂ is an electron, then the other is also an electron;
alternatively, R₁ and R₂ combine to form ═C(R₆)(R₇);
R₃ and R4 are each independently selected from the group: a bond to L, OH, C₁₋₁₀ alkyl substituted with 0-3 R₅, aryl substituted with 0-3 R₅, C₃₋₁₀ cycloalkyl substituted with 0-3 R₅, heterocyclo C₁₋₁₀ alkyl substituted with 0-3 R₅, wherein the heterocyclo group is a 5-10 membered heterocyclic ring system containing 1-4 heteroatoms independently selected from N, S, and O, C₆₋₁₀ aryl-C₁₋₁₀ alkyl substituted with 0-3 R₅, C₁₋₁₀ alkyl-C₆₋₁₀ aryl substituted with 0-3 R₅, and a 5-10 membered heterocyclic ring system containing 1-4 heteroatoms independently selected from N, S, and O and substituted with 0-3 R₅;
R₅ is independently selected at each occurrence from the group: a bond to L, ═O, F, Cl, Br, I, CF₃, CN, CO₂R₈, C(═O)R₈, C(═O)N(R₈)₂, CHO, CH₂OR₈, OC(═O)R₈, OC(═O)OR₈ₐ, OR₈, OC(═O)N(R₈)₂, NR₉C(═O)R₈, NR₉)C(═O)OR₈ₐ, NR₉C(═O)N(R₈)_{2 , NR}₉SO₂N(R₈)₂, NR₉SO₂R₈ₐ, SO₃H, SO₂R₈ₐ,, SR₈, S(═O)R₈ₐ, SO₂N(R₈)₂, N(R₈)₂, NHC(═S)NHR₈, ═NOR₈, NO₂, C(═O)NHOR₈, C(═O)NHNR₈R₈ₐ, OCH₂CO₂H, 2-(1-morpholino)ethoxy, C₁₋₅ alkyl, C₂₋₄ alkenyl, C₃₋₆ cycloalkyl, C₃₋₆ cycloalkylmethyl, C₂₋₆ alkoxyalkyl, aryl substituted with 0-2 R₈, and a 5-10 membered heterocyclic ring system containing 1-4 heteroatoms independently selected from N, S, and O;
R₈, R₈ₐ, and R₉ are independently selected at each occurrence from the group: a bond to L, H, C₁₋₆ alkyl, phenyl, benzyl, C₁₋₆ alkoxy, halide, nitro, cyano, and trifluoromethyl;
Pg is a thiol protecting group;
R₆ and R₇ are independently selected from the group: H, C₁₋₁₀ alkyl, CN, CO₂R₁₀, C(═O)R₁₀, C(═O)N(R₁₀)₂, C₂₋₁₀ 1-alkene substituted with 0-3 R₁₁, C₂₋₁₀ 1-alkyne substituted with 0-3 R₁₁, aryl substituted with 0-3 R₁₁, unsaturated 5-10 membered heterocyclic ring system containing 1-4 heteroatoms independently selected from N, S, and O and substituted with 0-3 R₁₁, and unsaturated C₃₋₁₀ carbocycle substituted with 0-3 R₁₁;
alternatively, R₆ and R₇, taken together with the divalent carbon radical to which they are attached form: 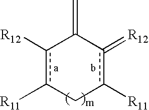
a and b indicate the positions of optional double bonds and m is 0 or 1;
R₁₁ and R₁₂ are independently selected from the group: H, R₁₃, C₁₋₁₀ alkyl substituted with 0-3 R₁₃, C₂₋₁₀ alkenyl substituted with 0-3 R₁₃, C₂₋₁₀ alkynyl substituted with 0-3 R,₁₃, aryl substituted with 0-3 R₁₃, a 5-10 membered heterocyclic ring system containing 1-4 heteroatoms independently selected from N, S, and O and substituted with 0-3 R₁₃, and C₃₋₁₀ carbocycle substituted with 0-3 R₁₃;
alternatively, R₁₁ and R₁₂ taken together form a fused aromatic or a 5-10 membered heterocyclic ring system containing 1-4 heteroatoms independently selected from N, S, and O;
R₁₃ is independently selected at each occurrence from the group: ═O, F, Cl, Br, I, CF₃, CN, CO₂R₁₀, C(═O)R₁₀, C(═O)N(R,₁₀)₂, N(R₁₀)₃₊, CH₂OR₁₀, OC(═O)R₁₀, OC(═O)OR₁₀ₐ, OR₁₀, OC(═O)N(R₁₀)₂, NR₁₄C(═O)R₁₀, NR₁₄C(═O)OR₁₀ₐ, NR₁₄C(═O)N(R₁₀)₂, NR₁₄SO₂N(R₁₀)₂, NR₁₄SO₂R₁₀ₐ, SO₃H, SO₂R₁₀ₐ, SR₁₀, S(═O)R₁₀ₐ, SO₂N(R₁₀)₂, N(R₁₀)₂, NOR₁₀, C(═O)NHOR₁₀, OCH₂CO₂H, and 2-(1-morpholino)ethoxy; and,
R₁₀, R₁₀ₐ, and R₁₄ are each independently selected at each occurrence from the group: hydrogen and C₁₋₆ alkyl;
or a pharmaceutically acceptable salt thereof.

23. A method of imaging myocardium perfusion, comprising administering the contrast agent of claim 1 to a patient and scanning the patient using diagnostic imaging.

24. The method of claim 23, wherein the imaging comprises at least one of MRI imaging or ultrasound imaging.

25. A contrast agent comprising:
a liquid perfluorocarbon encapsulated by a lipid composition, the lipid composition comprising:
i) a composition of the formula A-B, wherein A is a lipid or lipophilic moiety and B is a negatively charged component;
ii) an architectural lipid; and
iii) a composition of the formula A-Ch wherein Ch is a chelator, with the proviso that the contrast agent has an overall negative charge.

26. A method of imaging myocardium perfusion, comprising administering the contrast agent of claim 25 to a patient and scanning the patient using diagnostic imaging.

27. The method of claim 26, wherein the imaging comprises at least one of MRI imaging or ultrasound imaging.

28. A contrast agent comprising:
a gaseous perfluorocarbon encapsulated by a lipid composition, the lipid composition comprising:
i) a composition of the formula A-B, wherein A is a lipid or lipophilic moiety and B is at least one of carboxylic acid, tetrazole, boronic acid, phosphonic acid, phosphinic acid, sulfonic acid, or a compound of the Formula I: 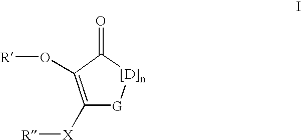
wherein:
n is 0, 2, or 3;
D is C(═O);
G is a bond or C(OH)═C(OH);
R′ is H, a pharmaceutically acceptable cation, or a bond to said lipid or lipophilic moiety;
R″ is H, or a bond to said lipid or lipophilic moiety; and X is O, S, NR′″, or C(R′″)₂, wherein R′″ is, independently, H or C1-C6 alkyl;
ii) an architectural lipid; and
iii) a composition of the formula A-Ch wherein Ch is a chelator, with the proviso that the contrast agent has an overall negative charge.

29. A method of imaging myocardium perfusion, comprising administering the contrast agent of claim 28 to a patient and scanning the patient using diagnostic imaging.

30. The method of claim 29, wherein the imaging comprises at least one of MRI imaging or ultrasound imaging.
